(19) United States
(12) Patent Application Publication
(10) Pub. No.: US 2017/0296186 A1
(43) Pub. Date: Oct. 19, 2017

(54) SURGICAL FIXATION DEVICES, SYSTEMS, AND METHODS

(71) Applicant: BRADLEY P. BENGTSON, GRAND RAPIDS, MI (US)

(72) Inventor: BRADLEY P. BENGTSON, GRAND RAPIDS, MI (US)

(21) Appl. No.: 15/637,480

(22) Filed: Jun. 29, 2017

Related U.S. Application Data
(63) Continuation of application No. 13/547,712, filed on Jul. 12, 2012, now abandoned.
(60) Provisional application No. 61/506,955, filed on Jul. 12, 2011.

(51) Int. Cl. A61B 17/072; A61B 17/064; A61F 2/12; A61B 17/068; A61B 17/00

(52) U.S. Cl. CPC A61B 17/07207; A61B 17/0682; A61B 17/07292; A61B 17/0644; A61F 2/12; A61B 2017/00004; A61B 2017/00792; A61B 2017/00867; A61B 2017/0645; A61B 2017/0649; A61B 2017/00796

(57) ABSTRACT

A surgical fixation method including use of a fixation device and an introducer.

### RELATED APPLICATIONS

This application is a continuation of co-pending U.S. application Ser. No. 13/547,712 filed 12 Jul. 2012, which claims the benefit of Provisional application Ser. No. 61/506,955, filed 12 Jul. 2011.

### FIELD OF THE INVENTION

The invention generally relates to surgical stapling devices, systems, and methods for securing biological material, naturally occurring compounds or synthetic meshes for reconstructive or cosmetic surgery, abdominal wall reconstruction or hernia repair, and general and other surgical specialty repairs.

### BACKGROUND OF THE INVENTION

Breast reconstruction and revision following cosmetic surgery to the breast involves rebuilding, revising, supporting and reconstruction of a breast, usually in women. It involves using autologous, allogenic, alloplastic tissue or prosthetic, synthetic material to help restore or construct a natural-looking restored anatomical breast shape and volume.

There are many methods for breast reconstruction and revision available to patients today. One of the most common methods involves the use of a tissue expander and a subsequent breast implant along with an acellular dermal matrix (“ADM”) of human or animal nature or synthetic mesh material. In method, the surgeon inserts a tissue expander, a temporary silastic expandable implant, beneath a pocket, typically under the pectoralis major muscle on the chest wall. Typically, this chest muscle and breast skin must be expanded over a period of several weeks to create a pocket of sufficient size to hold the permanent implant.

Alternatively, some surgeons use a “direct-to-implant” or “one-step reconstruction” approach. In this method, the surgeon sutures varying sizes of acellular dermal matrix, or other autologous tissues, or synthetic meshes or processed natural materials such as silk, as an extension of the pectoralis muscle, or under the muscle or alternatively on top of the muscle between it and the skin usually down to the inframammary fold an outer chest wall. Acellular dermal matrix (“ADM”), as used herein, is understood to mean human or porcine tissue dermis that has been processed in such a way that the cellular elements and matrix remain intact, while removing other cellular components that may lead to both rejection or infection. Commercially available ADM derived from donated human tissue includes that sold under the ALLODERM®, DERMAMATRIX®, ALLOMAX®, and TUTOPLAST® names, among others. Commercially available ADM derived from porcine dermis includes STRATTICE® tissue matrix, among others. Some synthetic mesh materials such as MARLEX™ or those manufactured under the PROLINE name are also available along with absorbable meshes such as VICRYL® or combinations of these. A new silk matrix, SFRISCAFFOLD® as well as others, are also on the horizon.

When used for breast reconstruction, the sutured-in-place matrix acts as an internal “hammock” or “sling” at the inferior portion of the breast, thereby supporting an implant, breast tissue or a combination of both implant and breast supporting the inferior breast pole. The sutured-in-place matrix may create a natural “sling” support and ptosis (sag) of the breast, while supporting the implant or residual breast in position. By suturing the matrix into its exact position of the breast margins, the surgeon is able to precisely recreate the contour for the reconstructed breast, especially at the inferior, medial, and lateral positions and help mitigate malposition or implant or breast displacement, and lower pole stretch of the breast soft tissues thereby minimizing migration of the implant. These tissues may also provide some additional covering over the implant, thereby decreasing visible implant wrinkling and rippling as well as decreasing the chance of capsular contracture or excessive scar formation around the implant. Thus revisions and complications are decreased.

The matrix materials described are increasingly used in breast revision following breast augmentation as well as in complex hernia repair and in new and emerging areas of nearly all areas of surgery.

Post-surgery, the ADM may then revascularize and integrate into the patient's healthy tissue, to thereby provide additional coverage over the implant. The use of ADM or meshes in “direct-to-implant” reconstruction make possible a single-step implant procedure that does not require a first expansion stage and, similar to a breast implant, revision allows the surgeon to use these materials as a muscle extension providing additional pocket coverage over the devices.

These procedures may involve endoscopic or laparoscopic surgery techniques, encompassing modern, or minimally invasive surgical procedures, in which access to the surgical field is gained through one or more relatively small incisions. Minimally invasive surgical procedures are desirable because they create less tissue disruption, less initial pain, less scarring and reduced post-operative patient discomfort; shortened recovery and hospitalization time; and reduced exposure of internal organs to possible contaminants.

There is also an intermediate type of procedure termed minimal access or limited access surgery may be compared to “operating through a mail slot.” Placement of sutures or staples to inset the ADM or mesh scaffold requires a suture or stapling device that is sharp and strong enough to pierce the matrix and underlying muscle, and to hold the matrix and underlying muscle tightly, yet can be ergonomically manipulated in the space and visualization-restricted environment of limited access or endoscopic incisions.

### SUMMARY OF THE INVENTION

The present invention provides surgical stapling devices and methods of use suited for use in surgical environments having limited space and restricted visualization and access. The technical features of the surgical stapling devices make possible unique surgical stapling systems and methods, e.g., for suturing ADM material, other biologics, natural based products or synthetic materials to the muscle, chest or abdominal wall or other deep tissues or along the inframammary fold of the breast, face and body.

The present invention further provides novel fixation systems. Fixation systems according to the present invention preferably include a fixation device, and an introducer.

Fixation devices for use with the present system may include: an absorbable barbed fixation device, staple, suture or band or strap; a nonabsorbable barbed fixation device, staple, suture band or strap; and a non-absorbable metallic or plastic staple, or circular ring device. An introducer may be further included in the present system to introduce a selected fixation device to a patient's tissue. An introducer for use according to the present system may include a novel, sharp “U”-shaped needle introducer through which the fixation device is guided and introduced, leaving in place the barbed fixation segment, or plastic, metallic clip. Alternatively, a corkscrew introducer could place and secure an ADM or mesh to the patient's soft tissues by spinning the device through both tissues in a manner similar to a spiral notebook, and leaving a retained barbed segment in position holding the tissues fixed together. An alternative permanent fixation clip made of titanium or other non-reactive metal may be placed through the “U”-shaped introducer that would penetrate the ADM or mesh and then soft-tissues that would then fold back on itself securing the mesh in position.

Additional designs provide a surgical fixation, stapling device that may include a memory or bias that urges the device to normally curve back upon itself to form a closed, generally circular ring after it is released or engaged. The device can be bent out of its normally closed condition by the application of an external opening force (e.g., applied by a companion applicator introducer) and into an opened, curvilinear shape when released. In the opened condition, the device includes a leading end and a trailing end. The leading end is sized and configured to penetrate the ADM or other material used in the reconstruction and underlying muscle or deep tissue, when the device is in its opened condition. The trailing end is sized and configured to transmit force to advance the leading end into tissues.

According to an aspect of the present invention, the fixation device can be progressively advanced through muscle or deep tissue as the external opening force is progressively removed. The memory or bias of the fixation device serves to progressively bend the device back toward its normally closed condition. The device moves progressively in an increasingly circular path through the muscle or deep tissue underlying the ADM, continuously curving back upon itself, in a direction toward and beneath the ADM. The leading end eventually exits the muscle or deep tissue a short distance from its original point of entry in the ADM. The fixation device eventually reforms as a tight loop or ring, which passes through the ADM and underlying muscle or deep tissue, thereby securing the ADM to the underlying muscle or deep tissue. This aspect of the present invention provides a surgical stapling fixation device that can be manipulated by a companion introducer tool in a simple and straightforward way in the space and visualization-restricted environment of endoscopy. The surgical fixation device and companion applicator tool make possible minimally invasive surgical systems and methods, e.g., for securing ADM to tissue in breast reconstruction surgery and the like.

A fixation device according to the present invention is preferably made of a stiff absorbable material such as polydioxanone (PDS or PDO), or poliglecaprone 25 (sold under the MONOCRYL® name) suture. Alternatively, the device may be made of a more permanent suture material, such as that sold under the PROLINE™ name, polypropylene suture, or other plastic or non-reactive metal that is biologically safe, non-reactive and also smooth to prevent damage to the implant or underlying body structures.

According to another aspect of the invention, the leading end of the fixation device may include a male locking component. The trailing end may include a female locking component sized and configured to receive and lock the male locking component therein when the device is in its normally closed condition. The ability to enclose a tissue-penetrating leading end entirely within a female locking component shields tissue, ADM, and the implant itself from incidental contact with and damage by the tissue-penetrating leading end. In addition, the metallic component may close back on itself after being introduced by a novel “U”-shaped introducer applicator.

The nature and scope of the invention may be best understood by reference to the following description, taken in conjunction with the accompanying drawings.

### BRIEF DESCRIPTION OF THE DRAWINGS

FIGS. 1A and 1B are perspective anatomic right lateral and left lateral views of an acellular dermal matrix (“ADM”) stitched to muscle or deep tissue in the chest by a plurality of surgical stapling devices that embody features of the invention, the stitched ADM serving as a “hammock” or “sling” at the inferior portion of the breast for support of an implant.

FIG. 1C is an enlarged view of a portion of the ADM illustrated in FIG. 1A, and showing further details of the plurality of surgical stapling devices that secure the ADE or other autologous or synthetic material to muscle or deep tissue.

FIG. 2 is a perspective view showing an inserter device having a handle and shaft for use with the present invention.

FIG. 3A is a view similar to that of FIG. 2, but showing the device in use with a fixation device exiting the shaft.

FIG. 3B is an enlarged view of a portion of the inserter device shaft shown in FIG. 3A with fixation device exiting the shaft.

FIG. 4 is a view similar to that of FIG. 3B, but showing an alternative fixation device.

FIG. 4A is similar to that of FIG. 4, showing another alternative fixation device.

FIG. 5 is a view similar to that of FIGS. 3B and 4, but showing an alternative fixation device.

FIG. 6A is a view similar to that of FIG. 2, but showing an alternative inserter device having a stationary handle and rotary knob driver.

FIG. 6B is an enlarged view of a portion of the inserter device shaft shown in FIG. 6A.

FIG. 7A is a view similar to that of FIG. 6A, but showing the inserter device in use with a fixation device exiting the shaft.

FIG. 7B is an enlarged view of a portion of the shaft with fixation device exiting as shown in FIG. 7A.

FIG. 8 is a perspective view of the inserter device illustrated in FIGS. 6A-7B, and showing the ejection button ejecting a fixation device.

FIG. 9A is a view similar to that of FIG. 6A, and showing an alternative embodiment inserter device having curved shaft exit.

FIG. 9B is an enlarged view of a portion of the inserter device shaft exit shown in FIG. 9A.

FIG. 10A. is a view similar to that of FIG. 9A, but showing the inserter device in use with a fixation device exiting the shaft.

FIG. 10B is an enlarged view of a portion of the inserter device shaft exit shown in FIG. 10A.

FIG. 11 is a perspective view of the inserter device illustrated in FIGS. 9A-10B, and showing the ejection button ejecting a fixation device.

FIGS. 12A and 12B are views showing an embodiment of a surgical fixation device that embodies features of the invention, FIG. 12A illustrates the device in an opened condition for placement into tissue and FIG. 12B depicts the fixation device in a closed condition once placed into tissue.

FIGS. 13A and 13B are views similar to those of FIGS. 12A and 12B, but showing another embodiment of a surgical fixation device that embodies features of the invention, FIG. 13A illustrates the device in an opened condition for placement into tissue and FIG. 13E depicts the device in a closed condition once placed into tissue.

FIGS. 14A and 14B are views similar to those of FIGS. 12A, 12B, 13 A, and 13B, but showing another embodiment of a surgical fixation device that embodies features of the invention, FIG. 14A illustrates the device in an opened condition for placement into tissue and FIG. 14B depicts the device in a closed condition once placed into tissue.

FIGS. 15A-15D are views illustrating the use of an inserter device to secure an ADM to muscle or deep tissue through use of a surgical fixation device such as that shown in FIGS. 12A and 12B.

FIG. 16 is a front view of another embodiment of a surgical fixation device that embodies features of the invention, with the surgical fixation device being shown in a closed position.

FIGS. 17A-17D illustrate another embodiment of a surgical fixation device that embodies features of the invention, with FIGS. 17B and 17D showing alternated positions of the fixation device in the closed position.

FIGS. 18A-18B show the surgical fixation device illustrated in FIGS. 17A-17D in use to secure to tissue.

FIG. 19 further demonstrates the surgical fixation device of FIGS. 17A-17D being used to secure a synthetic mesh to tissue.

FIGS. 20A and 20B are fragmentary views of an inserter device shaft and showing fixation device exit therethrough.

FIGS. 20C and 2CD are enlarged fragmentary views of embodiments of the surgical fixation device and showing various arrangement with a wire shown in FIG. 20C and a ribbon shown in FIG. 20D.

FIGS. 21A-21D illustrate a method of using the present system to suture ADM to muscle or deep tissue.

FIGS. 22A-22D are views similar to those of FIGS. 21A-21D, but showing an alternative method of using the present system to suture ADM to muscle or deep tissue.

### DESCRIPTION OF THE PREFERRED EMBODIMENT

Although the disclosure hereof is detailed and exact to enable those skilled in the art to practice the invention, the physical embodiments herein disclosed merely exemplify the invention, which may be embodied in other specific structure. While the preferred embodiment has been described, the details may be changed without departing from the invention, which is defined by the claims.

The present invention is directed toward surgical fixation systems. Fixation systems according to the present invention preferably include a fixation device and an introducer, and method of using as will be discussed.

The technical features of the invention are highlighted in the context of securing ADM to tissue in breast reconstruction surgery. It should be appreciated, however, that the technical features of the invention are well suited to surgical procedures in general, and, in particular, to minimally invasive surgical procedures, hernia surgery, or other endoscopic procedures.

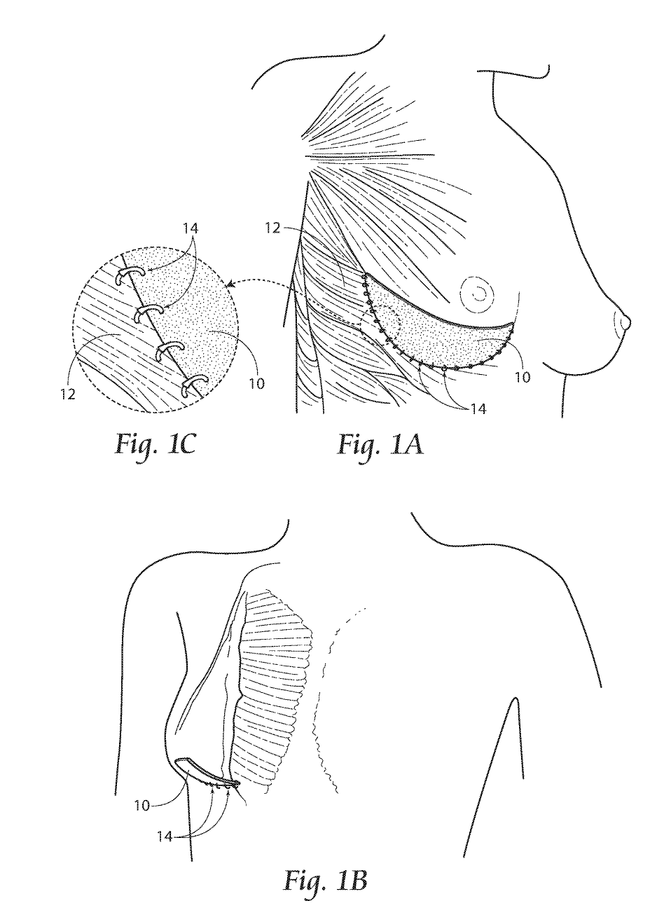

With reference now to the drawings and in particular to FIGS. 1A and 1B, an ADM 10 stitched to muscle or deep tissue 12 in the chest by a plurality of surgical fixation devices 14 that embody features of the invention may be seen. As mentioned previously, the sutured ADM 10 serves as a “hammock” or “sling” at the inferior portion of the breast for support of an implant, e.g., in breast revision or reconstruction surgery.

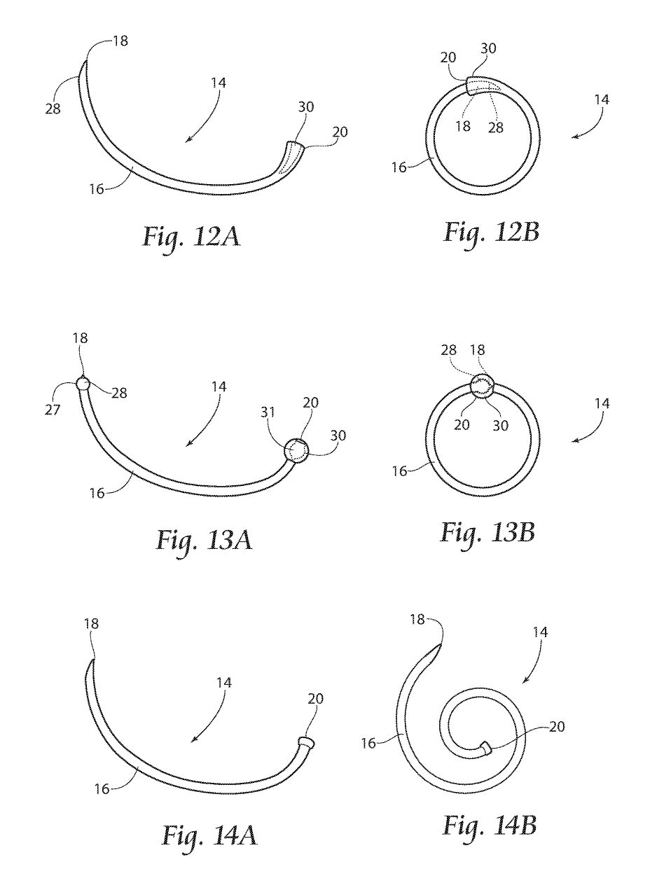

FIG. 1C is an enlarged view of a portion of the ADM 10 shown in FIG. 1A, but showing further details of the plurality of surgical fixation devices 14 that secure the ADM 10 to muscle or deep tissue 12. Still further details can also be seen in the views of FIGS. 12A and 12B.

As shown in the views of FIGS. 12A and 12B, each surgical fixation device 14 preferably includes a body member 16, The body member 16 can be formed of an insert extruded or molded plastic polymer or metallic material, or combinations thereof. Desirably, the material is bio-absorbable, meaning that it transforms over time within the body from a solid state to a state that can be cleared or absorbed by the body. Candidate bio-absorbable polymers include, polyglycolic acids, polyesters, poly(amino acide),polyanhydrides, polyorthoesters, polyurethanes, and polycarbonates among others.

The body member 16 includes a memory or bias that urges the body member 16 to normally curve back upon itself to form a closed, generally circular loop or ring, as shown in FIGS. 1C and 12B. This configuration will in shorthand be called the normally closed condition.

As shown in FIG. 12A, the body member 16 can be bent out of its normally closed condition by the application of an external force into an opened, curvilinear shape. This configuration will in shorthand be called the opened condition. When the external force is relied, the memory or bias or the body member 16 will return the body member 16 toward the normally closed condition, as is shown in FIG. 12B. In the opened condition, the curvilinear body member 16 presents a leading end 18 and a trailing end 20, as can be seen in FIG. 12A. The leading end 18 is sized and configured to penetrate tissue when the body member 16 is in its opened condition. The leading end 18 can include a sharpened or pointed edge, but need not. For example, the leading end 18 can be stiffened with a blunt edge to pass through muscle or deep tissue 12.

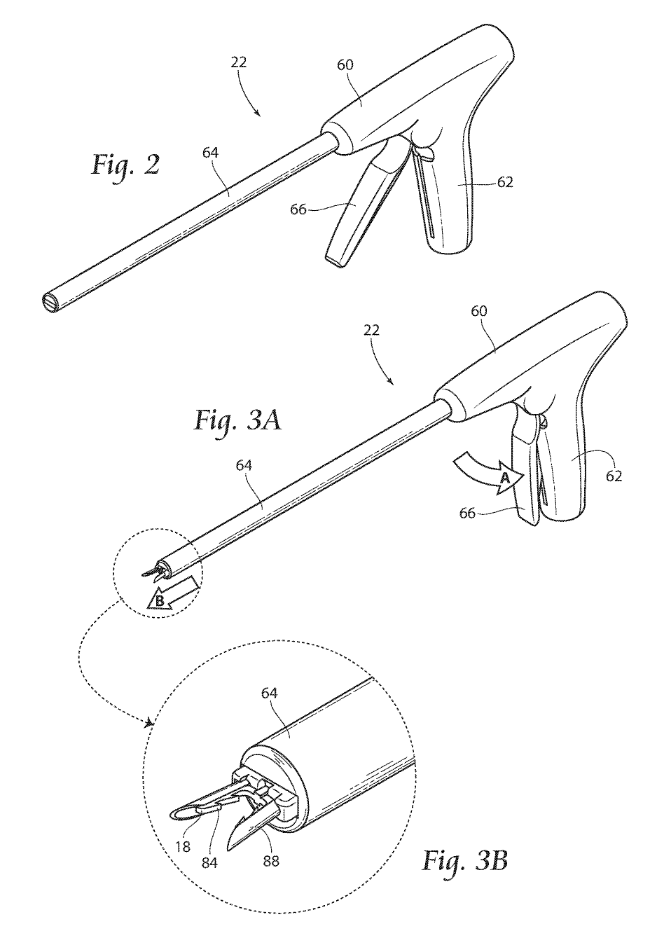

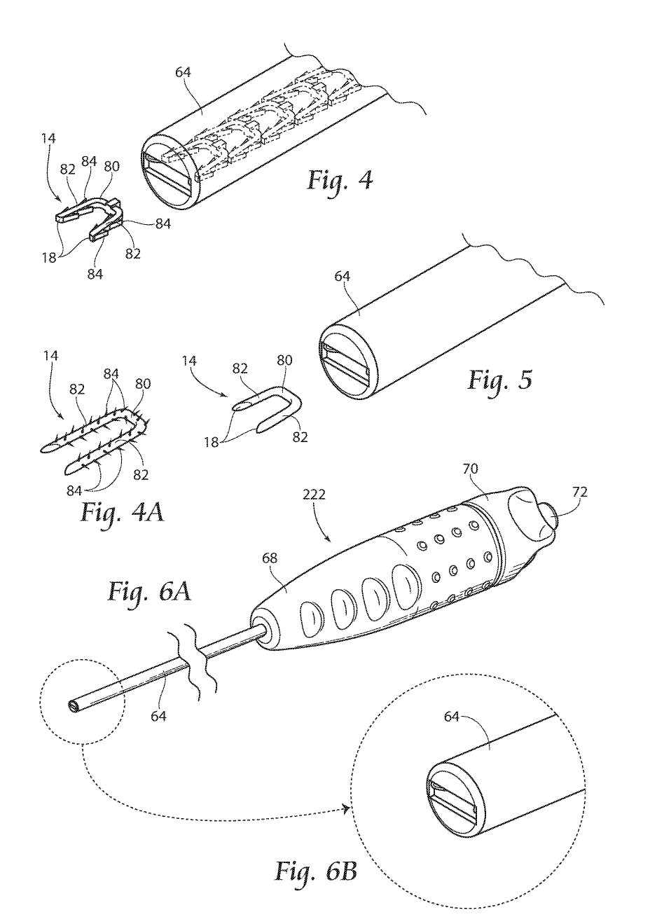

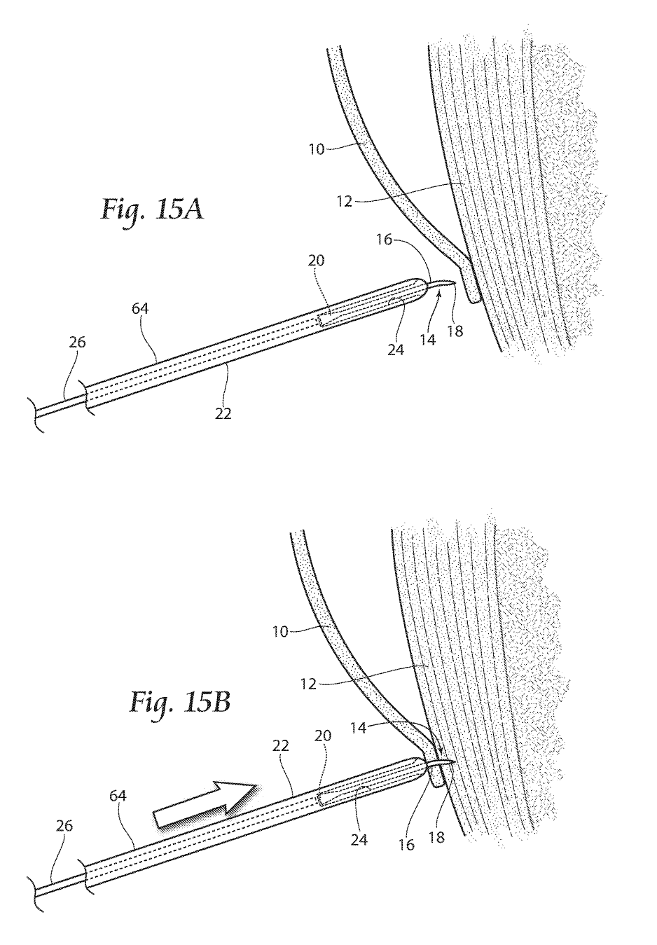

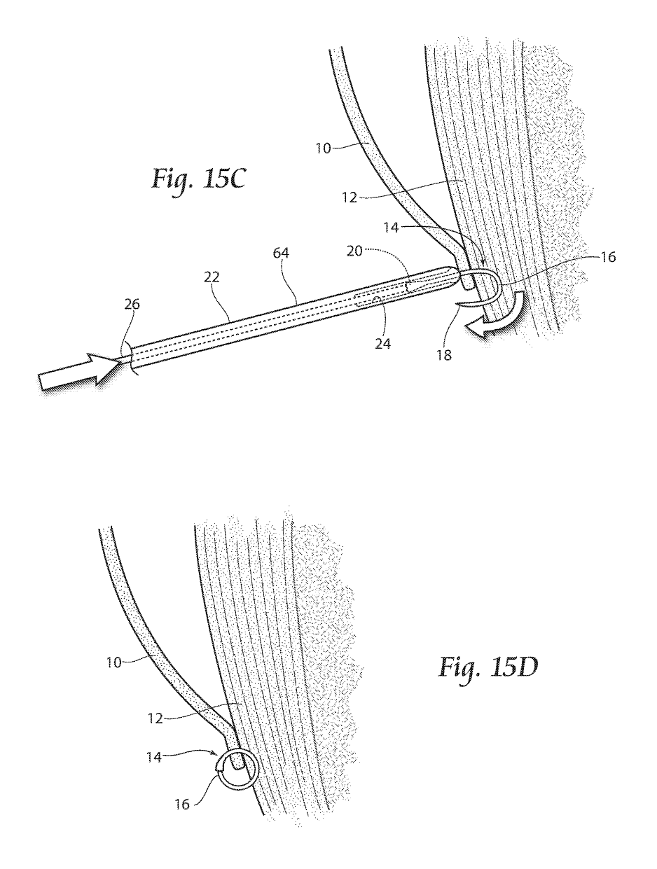

Referring to the drawings and particular to FIGS. 2 and 3A, an embodiment of an applicator tool or introducer, 22 incorporating the principles of the present invention is shown as having a housing 60 in the shape of a pistol or gun and having a handle or grip portion 62, a shaft portion 64, and a trigger 66. The trigger 66 is located forwardly of the grip 62 and under the shaft portion 64 where it fits naturally in the hand of a user (not shown). The introducer 22 is typically used to introduce fixation devices 14 (seen in FIGS. 12A-14B) to ADM 10 or other autologous tissues, synthetic meshes and the like, and to secure the ADM 10 to muscle or deep tissue 12. As mentioned earlier, ADM 10 is widely used in a variety of environments and applications, particularly breast reconstruction. The introducer 22 may assume varying configurations depending on the fixation device 14 to be deployed. FIGS. 2-5, for example, illustrate an introducer 22 having an interior channel 24 a plunger 26 (see FIGS. 15A-15C). As seen, as the trigger portion 66 is moved in the direction of arrow A, the plunger 26 presses the fixation device 14 in the direction of arrow B and toward the surgical area (not shown in these views). As seen, the particular fixation device 14 shown in FIGS. 3B and 4 may be of a generally u-shape having a curved portion 80 and two extending arm portions 82. The arm portions 82 each terminating in a leading end 18, although it is to be understood that any of the fixation devices disclosed herein may be deployed through use of the introducer 22 depicted. As further illustrated, the arms 82 may include a plurality of upstanding ridges or barbs 84 (see FIGS. 4 and 4A) to resist movement once and aid in fixation once in place. As shown in FIG. 4A, the arms 82 are extended in length compared to the arrangements of FIGS. 4 and 5, and the barbs 84 are preferably arranged in a bidirectional arrangement. Alternatively, and as viewed in FIG. 5, the arms 82 of fixation device 14 may be devoid of ridges. As seen, the fixation devices 14 may be stacked in the shaft 64 in a manner to allow multiple firing and advancing of devices 14 for user convenience. The plunger 26 of the introducer 22 may also include a u-shaped bracket 88 adapted to cradle the arms 82 of a fixation device 14 when the fixation device 14 used is so configured.

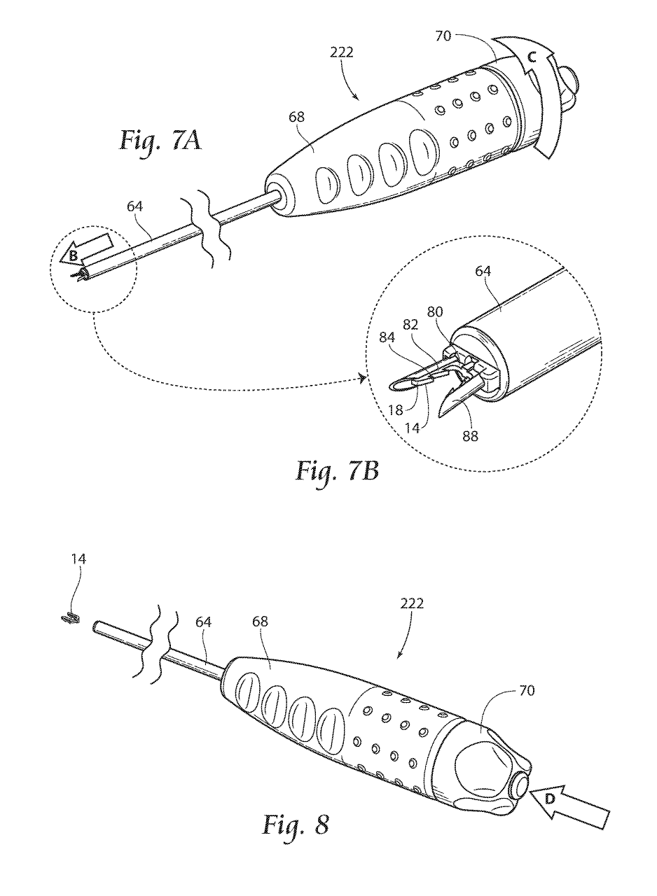

With reference now to the views of FIGS. 6A-8, an alternative introducer tool 222 and its use may be seen. Similar to the introducer 22 shown in FIGS. 2-5, the introducer 222 illustrated in FIGS. 6A-8 preferably includes a housing 60 and a shaft portion 64. However, rather than the trigger 66 of the previous embodiment, the introducer 222 illustrated in these views includes a stationary handle 68 and a rotary knob 70 to drive the plunger (not shown). Further, a fixation device ejection button 72 located on an end 73 of the introducer 222 and serves to deploy the fixation devices 14 from the shaft 64. As in the previous embodiment, the introducer 222 is used to introduce fixation devices 14 to ADM 10 or other autologous tissues, synthetic meshes and the like, and to secure the ADM 10 to muscle or deep tissue 12. As seen, as the rotary knob 70 is moved in the direction of arrow C, the plunger 26 presses the fixation device 14 in the direction of arrow B and toward the surgical area (not shown in these views). Pressing the ejection button 72 in the direction of arrow D deploys the fixation device 14. The particular fixation device 14 shown in FIGS. 7A-8 is similar to that shown in FIGS. 3A-4, and has a generally u-shape having a curved portion 80 and two extending arm portions 82. The arm portions 82 each terminate in a leading end 18, although it is to be understood that any of the fixation devices disclosed herein may be deployed through use of the introducer 22 depicted. As further illustrated, the arms 82 may include a plurality of upstanding ridges 84 to resist movement once and aid in fixation once in place. Alternatively, and as viewed in FIG. 5, the arms 82 of fixation device 14 may be devoid of ridges. The plunger 26 of the introducer 222 may also include a u-shaped bracket 26 adapted to cradle the arms 82 of a fixation device 14 when the fixation device 14 used is so configured.

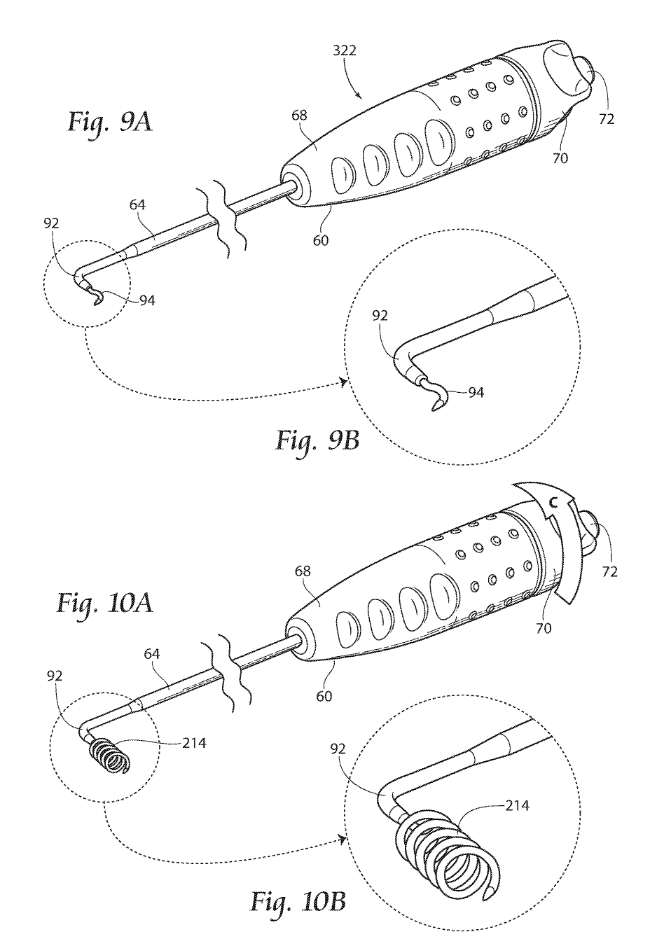

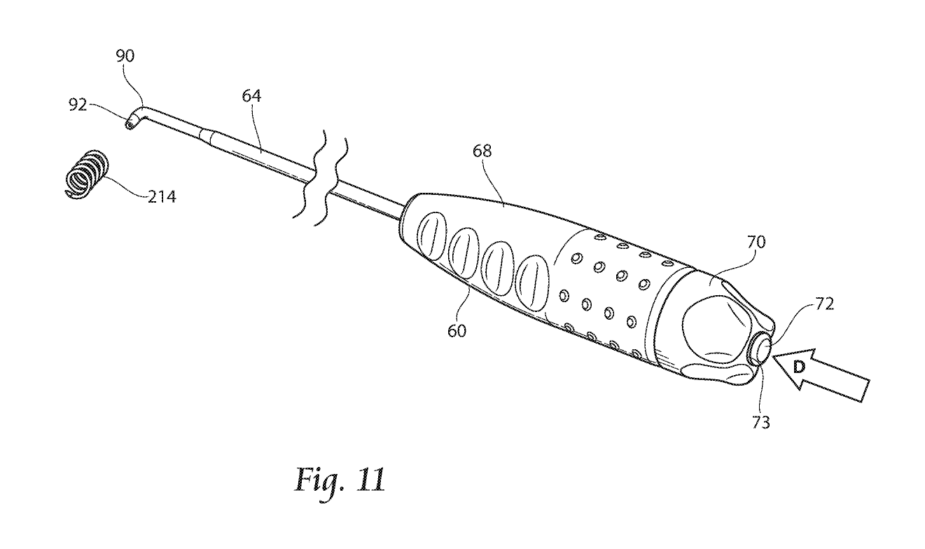

FIGS. 9A-11 illustrate an alternative introducer tool 322 and its use. Similar to the introducer 222 shown in FIGS. 6A-8 introducer 322 preferably includes a housing 60, a shaft portion 64, a stationary handle 68 and a rotary knob 70 to drive the plunger (not shown). As seen, the shaft portion 64 further includes distal end 90 having an angled portion 92. The angled portion 92 is used to deploy the corkscrew fixation devices 14 illustrated. A fixation device ejection button 72 is located on an end 73 of the introducer 322. As the rotary knob 70 is moved in the direction of arrow C, the plunger 26 presses the fixation device 14 in the direction of arrow B and toward the surgical area (not shown in these views). Pressing the ejection button 72 in the direction of arrow D deploys the fixation device 14. The particular fixation device 14 shown in FIGS. 9A-11 is unlike that shown in FIGS. 3A-4, and has a generally corkscrew shape. The use of a fixation device 14 having this configuration will be discussed below. As seen particularly in FIGS. 9A and. 9B, the plunger 26 may also include an angled portion 94 adapted to conform to the shaft angled portion 92.

With particular reference to the views of FIGS. 15A-15D, the shaft 64 of the applicator tool or introducer 22 is preferably sized and configured with an interior channel 24 to receive the fixation member 14 trailing end 20 and body member 16. The leading end 18 may project beyond the channel 24 for penetration of tissue. The confines of the channel 24 apply the external force necessary to hold the body member 16 in its opened condition.

As FIGS. 15B and 15C further show, the companion introducer 22 may include a plunger 26 that abuts against the trailing end 20 of fixation member 14. The plunger 26 is sized and configured for advancement axially through the channel 24. Axial advancement of the plunger 26 expels the body member 16 progressively outward of the channel 24, leading end 18 first. The leading end 18 of the body member 16 first penetrates the material of the ADM 10, and then progresses into underlying muscle or deep tissue 12 (see FIG. 15B). Openings or apertures (not shown) can be pre-formed along the periphery of the ADM 10 to aid in deployment of the surgical fixation devices 14 using the introducer 22.

Further advancement of the plunger 26 (see FIG. 15C) expels more of the body member 16 from the channel 24. Being progressively freed from the external force applied by the channel 24, the memory or bias of the body member 16 progressively bends the body member 16 toward its normally closed condition. As shown, the body member 16 moves progressively in an increasingly circinate path through the muscle or deep tissue 12 underlying the ADM 10, and continuously curving back upon itself in a direction toward and beneath the ADM 10. The leading end 18 eventually exits the muscle or deep tissue 12 a short distance from its original point or entry in the ADM 10, as FIG. 15C shows.

As seen in FIG. 15D, further advancement of the plunger 26 eventually expels the trailing end 20 from the channel 24. The body member 16 is free to fully return to its normally closed condition. The body member 16 then reforms as a tight loop through the ADM 10 and underlying muscle or deep tissue 12, securing the ADM 10 to the underlying muscle or deep tissue 12.

The fixation device 14 is manipulated by the companion introducer tool 22 by the ergonomic application of simple, axial force in the space and visualization-restricted environment of endoscopy. As viewed particularly in the views of FIGS. 1C and 12B, the size and configuration of the fixation device 14 leading end 18 may also form a male locking component 28. Further, the fixation device 14 trailing end 20 may preferably include a female locking component 30 sized and configured to receive the male locking component 28 in sliding interference fit when the body member 16 is in its normally closed condition. This can be seen particularly in the views of FIGS. 1C and 12B. The ability to enclose the tissue-penetrating leading end 18 entirely within the female locking component 30 shields tissue, the ADM 10, and the implant itself from incidental contact with the tissue-penetrating leading end 18.

The technical features of the surgical fixation device 14 can be realized in other structural forms. For example, similar to the previously described surgical fixation device 14, the device 14 illustrated in FIGS. 13A and 13B, includes a memory or bias toward a normally closed condition, and can be placed in an opened condition as previously described, and with the same function as previously described. However, the embodiment shown in FIGS. 13A and 13B illustrates the male locking component 28 of the leading end 18 as including a ball-shaped protuberance 27. Accordingly, the female locking component 30 of the trailing end 20 includes a mating socket 31 that is sized and configured to receive the ball-shaped protuberance 27 of the male locking component 28 in a sliding snap-fit when the body member 16 is in its normally closed condition. This can be seen in FIG. 13B.

As another example, the body member 16 of the surgical fixation device 14 can comprise a spring-like material that is sized and configured to normally assume a helically coiled. or spiral condition corresponding to the previously described normally closed condition, as is shown in FIG. 14B. The material can be stretched by the application of tension from the normally coiled condition to a curvilinear configuration corresponding to the previously described opened condition, as is shown in FIG. 14A. The companion introducer tool 22 applies this tension to accommodate deployment of the body member 16, leading end 18 first. Progressive removal of the tensioning force, by expelling the body member 16 from the confines of an applicator tool 22, capes the body member 16 to progressively return to its normal helically coiled or spiral condition, securing the ADM 10 to the underlying muscle or deep tissue 12.

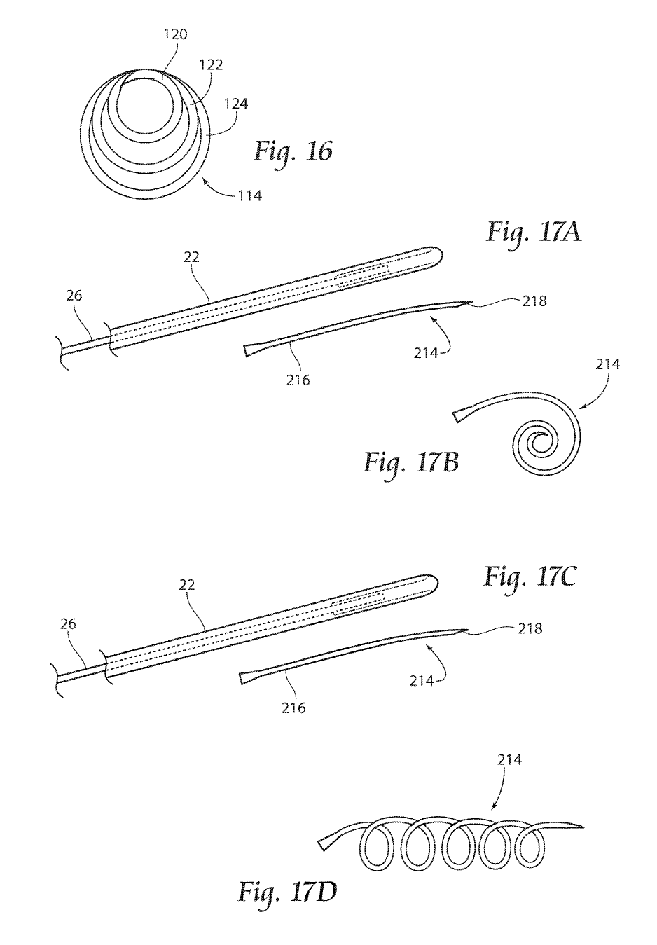

Referring now to FIG. 16, an alternative fixation device 114 formed according to the present invention may be seen. The fixation device 114 is delivered with an introducer tool 22 as previously described (see FIGS. 15A-15B). When delivered into tissue, the fixation device 114 will coil around itself 360° or more, forming a plurality of nesting circular loops, e.g. circular sections 120, 122, 124, that will allow the stapling device 114 to provide a plurality of loops for intersecting tissue and securing the fixation device 114 to the tissue, similarly as demonstrated with the device 14 previously described.

FIGS. 17A-17D demonstrate a further embodiment 214 of a fixation device according to the present invention. The device 214 is designed to act similarly to the fixation device 114, wherein the device 214 is capable of coiling around itself once it is inserted into tissue. The device 214 will be delivered by way of an introducer 22, similarly to the device 14 described previously. As seen, the device 214 generally comprises a straight, elongated body 216 with a leading end 218, which is preferably sharpened so that it can pierce tissue (not shown in these views) once it is inserted. The device 214 is designed so that it can coil around itself, as shown in FIG. 17B, or alternatively, can spiral along a central axis when secured to the tissue. Both final arrangements shown in FIGS. 17B and 17D allow for the fixation device 214 to provide a secured connection between the device 214 and the intersected tissue, as previously described.

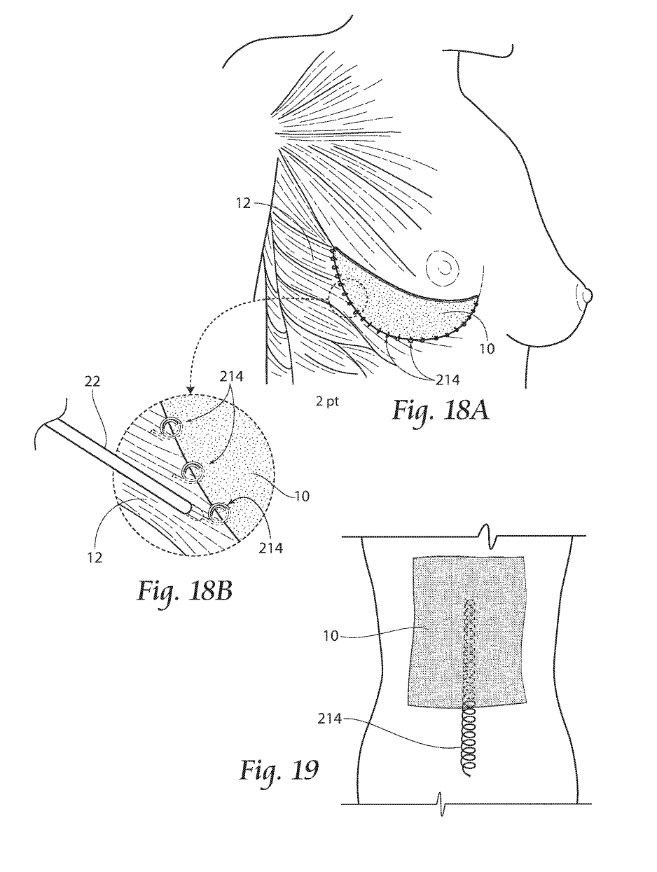

With reference now to FIGS. 18A and 18B, the stapling device 214 as demonstrated in FIGS. 17A-17B is shown securing muscle tissue 12 to an ADM 10 or mesh material by way of the fixation device or devices 214. As shown, the introducer 22 is inserted into the tissue 12 and the ADM 10, with the introducer 22 intended to pierce both the tissue 12 (e.g. muscle) and the ADM 10. Once properly positioned, the fixation devices 214 can be released as previously described with respect to the fixation device 14, to secure the muscle 12 and the ADM 10. As shown in FIG. 18B, the fixation device 214 allows for various positions thereof, to secure the muscle 12 and the ADM 10, with each of the positions being reinforced with multiple piercings or intersections located at each of the positions.

As previously noted, the fixation device 214 can also be delivered along a central axis so that it can form a spiraled spine shape for securing a mesh or ADM 10 to tissue or muscle. FIG. 19 shows such an arrangement. The fixation device 214, when inserted into the muscle 12 and the mesh, forms a continuous wound spiral, which may be useful in surgical procedures that require more fixation devices 14 to cover a larger area. An example of such a procedure may be the attachment of a mesh when performing a hernia operation or a surgical procedure around the abdominal wall. It should be understood that the fixation devices 14, 114, 214 of the present invention may be used with various surgical procedures.

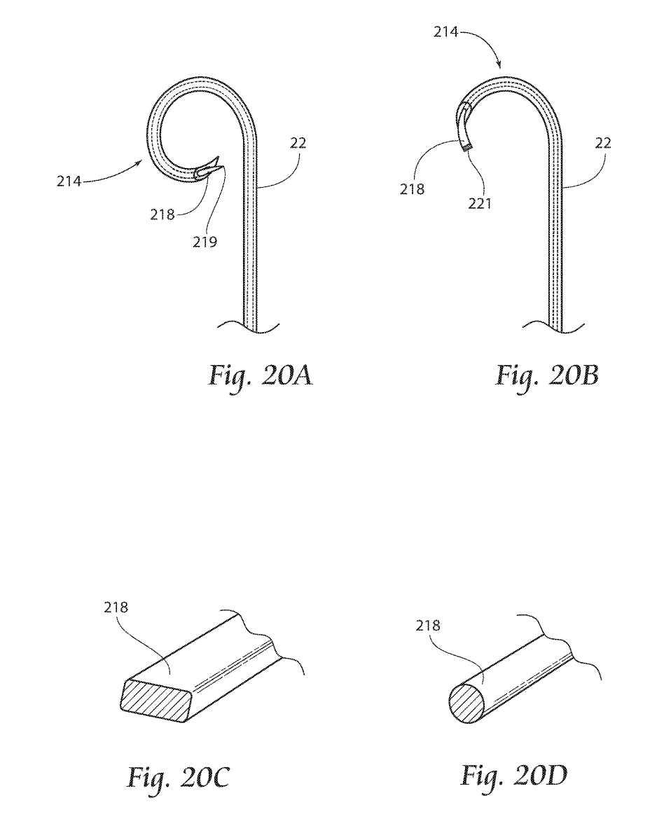

FIGS. 20A-20D illustrate various arrangements and delivery processes of the fixation device 214. As mentioned, the fixation device 214 is generally delivered with the use of an introducer 22. The introducer 22 as described in FIGS. 20A-20D preferably includes a hollow shaft 64, needle or hook, with the fixation device 214 being delivered through the introducer 22. FIG. 20A differs from FIG. 20B in that the fixation device 214 is illustrated as made of various materials, such as the wire shown in FIG. 20A or the ribbon illustrated in Figure FIG. 20B.

The fixation device 214 of FIG. 20A may be made of a shape retaining material and, also a coiled structure, as previously described, such as a nickel titanium alloy. One such common alloy is referred to as Nitinol. The end 218 of device 214 is shown in FIG. 20A as providing a sharpened point 219. However, as shown in FIG. 20C, the end 218 of the fixation device 214 may also have a blunt end 221, provided it is sufficiently designed to intersect the tissue or muscle with which the fixation device 214 is being attached.

FIG. 20D illustrates an end 218 of a fixation device 214, such as that shown in FIG. 20B, wherein the fixation device 214 comprises a ribbon as opposed to the wire shown in FIGS. 20A and 20C. A Nitinol material may also be used to form the ribbon or band form illustrated in FIGS. 20B and 20D. Depending on the particular surgical operation, a ribbon arrangement may be preferable to a wire arrangement. It is understood that such a fixation device 214 designed as described and shown in would fail within the scope of the present invention.

As previously discussed, the fixation devices 14, 114, 214 according to the present invention may be formed of a biocompatible material. Preferred material may include a metal alloy or metal material, with a shape retaining material being preferable, such as a Nitinol or NiTi material.

Depending on the particular surgical procedure being performed and/or the introducer device 22, 222, 322 being used, the fixation devices 14, 114, 214 of the present invention can be delivered individually or in a “multiple fire” fashion, wherein the introducer 22 can deliver one or more than one individual fixation devices 14, 214, 214 during use.

It is to be understood that fixation devices 14, 114, 214 according to the present invention may or may not have a sharpened leading end 18, depending on the particular use and arrangement of the fixation device. It is understood that either of such an arrangement will fall within the scope of the present invention, provided that the fixation device provides a securing arrangement of the tissue or muscle and the ADM 10, as discussed above.

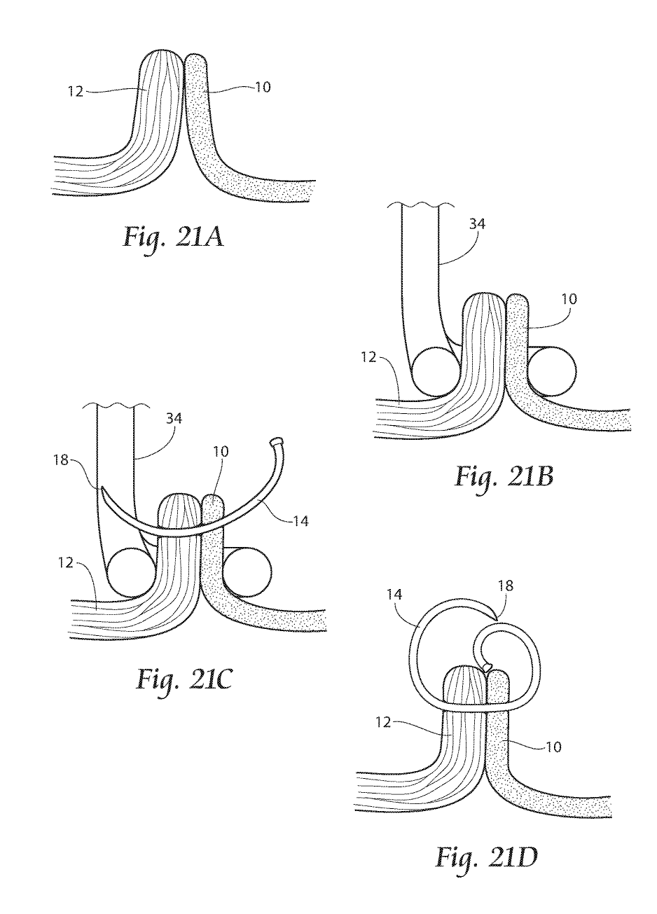

The views of FIGS. 21A-21D illustrate a method of using the present system to suture ADM 10 to muscle or deep tissue 12. As seen the ADM 10 and tissue 12 may be temporarily held in place by a clamp 34 or other known surgical means. The leading end 18 of a fixation device 14 according to the present invention is delivered by an introducer 22, 222, 322 (not seen in these views) and passed through the ADM 10 and tissue 12. The fixation device 14 then coils around itself to thereby secure the ADM 10 and tissue 12 to one another. Alternatively the fixation device 14 may secure the ADM 10 and tissue 12 through arrangements described earlier.

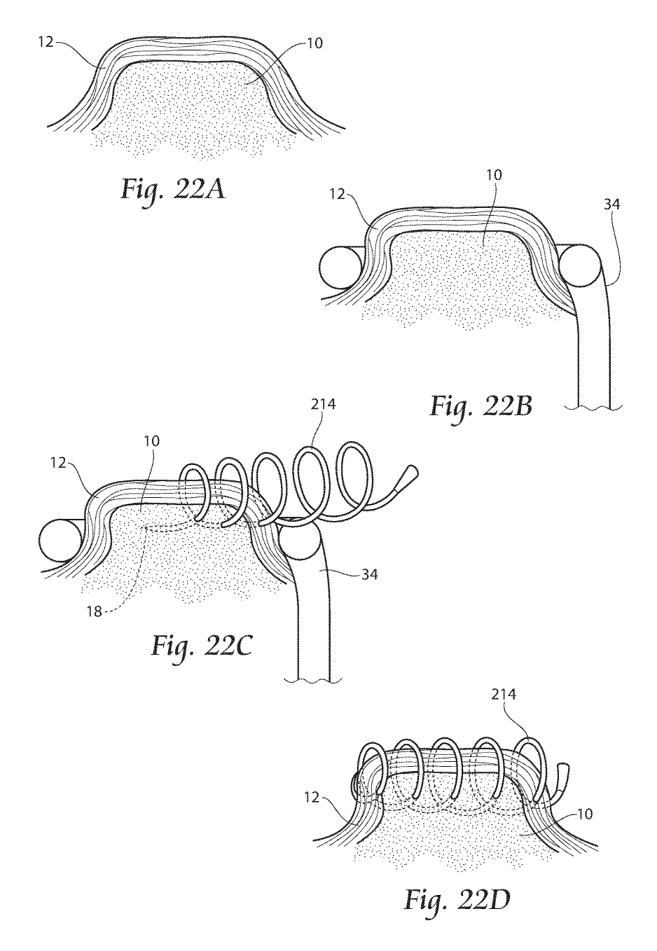

FIGS. 22A-22D are views similar to those of FIGS. 21A-21D, but showing method of using the present system to suture ADM 10 to tissue 12 using a spiral fixation device 214. As seen, and similar to the views of FIGS. 21A-21D, the ADM 10 and tissue 12 may be temporarily held in place by a clamp 34 or other known surgical means. The leading end 18 of a fixation device 214 according to the present invention is delivered by an introducer 22, 222, 322 (not seen in these views) and passed through the ADM 10 and tissue 12. The fixation device 214 may be delivered along a central axis to form a spiraled spine shape for securing the ADM 10 to tissue 12. The fixation device 214 forms a continuous wound spiral.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention.

I claim:

1. A method of suturing including the steps of:
providing a first tissue and a second tissue for suturing to said first tissue;
providing a surgical fixation system, said surgical fixation system including a fixation device and an introducer, said fixation device having a body member and at least one leading end, said introducer including a shaft portion, said shaft portion including a channel, and a plunger, said plunger located in said channel;
inserting the fixation device in said channel of said introducer;
locating said first and second tissues in an adjacent, suturable position;
moving said shaft to a position proximate said first and second tissues; and
actuating said plunger to drive said fixation device into said first and second tissues.

2. The method of claim 1 further the step of providing said fixation device with a u-shape having a curved portion and two extending arm portions, the arm portions each terminating in a leading end.

3. The method of claim 2 further including the step of providing at least one of said arm portions with a plurality of upstanding ridges.

4. The method of claim 1 further including the step of providing said body member with a bias memory that urges the body member to normally curve back upon itself to form a closed, generally circular loop.

5. The method of claim 4 further including the step of providing said fixation device leading end with a male locking component.

6. The method of claim 5 further including the step of providing said fixation device with a trailing end, said trailing end including a female locking component sized and configured to receive the male locking component in sliding interference fit.

7. The method of claim 6 further including the step of providing said male locking component with a ball-shaped protuberance and said female locking component with a mating socket that is sized and configured to receive the ball-shaped protuberance of said male locking component in a sliding snap-fit.

8. The method of claim 1 further including the step of providing said fixation device with a spring-like material that is sized and configured to normally assume a helically coiled condition.

9. The method of claim 1 further including the step of providing said introducer with a trigger member, said trigger member moveable between a first position and a second position, whereby movement of said trigger actuates said plunger to thereby deploy said fixation member from said channel.

10. A method of suturing including the steps of:
providing an acellular dermal matrix for suturing to a human. tissue;
providing a surgical fixation system, said surgical fixation system including a fixation device and an introducer, said fixation device having a body member and at least one leading end, said introducer including a shaft portion, said shaft portion including a channel, and a plunger, said plunger located in said channel;
inserting the fixation device in the channel of said introducer;
locating said acellular dermal matrix and said human tissue in an adjacent, suturable position;
moving said shaft to a position proximate said first and second tissues; and
actuating said plunger to drive said fixation device into said first and second tissues.

11. The method of claim, in which the step of locating further includes holding said acellular dermal matrix and said human tissue in an adjacent, suturable position using a clamping force.
